(19) United States
(12) Patent Application Publication
(10) Pub. No.: US 2019/0321503 A1
(43) Pub. Date: Oct. 24, 2019

(54) FLOOR MAT FOR SANITIZING SHOES

(71) Applicant: Terry Warnell, Tyler, TX (US)

(72) Inventor: Terry Warnell, Tyler, TX (US)

(21) Appl. No.: 15/961,563

(22) Filed: Apr. 24, 2018

(51) Int. Cl. A61L 2/10; A61L 2/26

(52) U.S. Cl. CPC A61L 2/10; A61L 2202/11; A61L 2/26

(57) ABSTRACT

A mat for sanitizing soles of shoes that includes: a top surface; a plurality of grooves along the top surface; a UV light source within each groove; a first side; a second side; and a bottom surface. In one particular embodiment, the first side and second side are beveled. Further each groove may be beveled to receive the UV light source. The present invention further relates to a method for sanitizing soles of shoes the includes the steps of: creating groove openings in a mat; inserting a UV light source in each groove; stepping onto the mat with a target pair of shoes; activating the UV light source while on the mat; and remaining on the mat for predetermined time period.

### BACKGROUND OF THE INVENTION

### Field of Invention

The present invention relates to a mat that is utilized to sanitize a pair of shoes.

### Description of Related Art

Floor mats are used frequently to collect or remove debris from the soles of shoes. Further, mats are used to absorb moisture that may accumulate on the shoe sole. Mats essentially have various types of application, such as floor mats are used in the bathrooms and showers. The typical floor mat is used in a doorway entrance either on the outside or inside thereof to allow individuals to remove debris from their shoes before entering the dwelling. Although the mat may effective remove larger macro debris, micro organisms will still exist on the sole of the shoe. Many times these micro organisms are in the form of bacteria and germs. Further, the shoe may appear clean but still may contain a considerable amount of bacteria and germs on the sole of the shoe due to exposure to certain conditions. As a result, it would be advantageous to have an effective mat that was able to remove and kill bacteria and germs from the surface of the shoe sole. Such a device could be effective in reducing the proliferation of bacteria and germs into a dwelling.

### SUMMARY OF THE INVENTION

The present invention relates to a mat for sanitizing soles of shoes that includes: a top surface; a plurality of grooves along the top surface; a UV light source within each groove; a first side; a second side; and a bottom surface. In one particular embodiment, the first side and second side are beveled. Further each groove may be beveled to receive the UV light source.

The present invention further relates to a method for sanitizing soles of shoes the includes the steps of: creating groove openings in a mat; inserting a UV light source in each groove; stepping onto the mat with a target pair of shoes; activating the UV light source while on the mat; and remaining on the mat for predetermined time period.

### BRIEF DESCRIPTION OF DRAWINGS

FIG. 1 depicts a top view of a floor mat in accordance with the present invention.

FIG. 2 depicts a sectional view of the floor mat in accordance with the present invention.

### DETAILED DESCRIPTION

The present invention relates to a floor mat that is used to sanitize the sole of a shoe. The floor mat includes a top surface and has a series of grooves along the surface. A UV light source is positioned within each groove. The UV light source provides a means to remove bacteria and germs from the sole of the shoe. Preferably, a user steps onto a mat for a predetermined period of time, usually a matter of seconds, and the UV light is activated while standing on the mat. Once this light is activated, the process of disinfecting the sole surface is initiated.

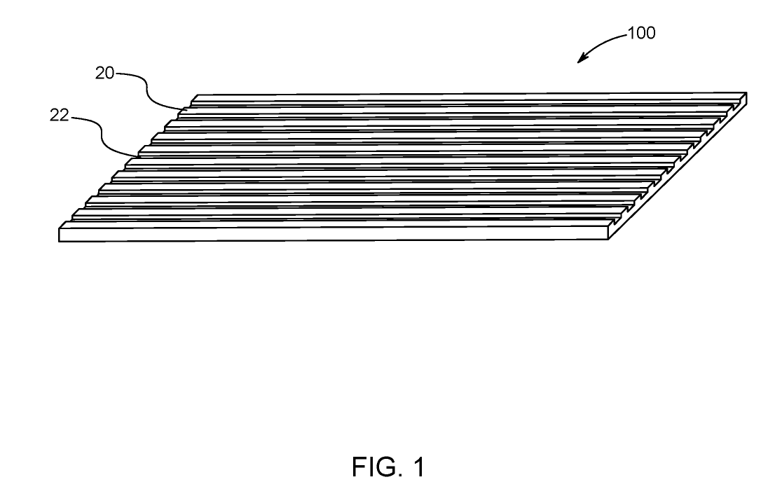

In reference to FIG. 1, a top view of the floor mat according to the present invention depicts a floor mat 100 that includes a plurality of grooves 22 along the longitudinal length of the mat 100. A top surface 20 provides the surface area for placement of an individual's shoes. Ideally, an individual steps onto the mat 100, where the act of stepping onto the mat activates the UV light source within the grooves. Once activated, the light source sanitizes the soles of the shoes in a matter of seconds.

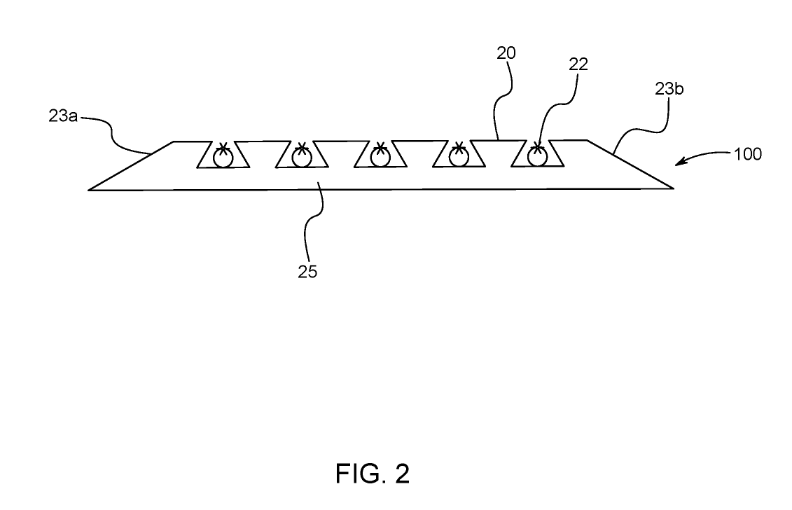

FIG. 2 depicts a cross section of mat 100 according to the present invention. This cross section depicts a view of each groove 22 as shown. In each groove 22 includes a UV light source. The mat 100 further includes the top surface 20 and a bottom surface 25 which is a flat, congruent surface. It is noted in this particular embodiment that each lateral side of the mat 100 is beveled as shown with lateral sides 23a, 23b. As a result, the mat 100 provides a method for sanitizing the soles of a shoe.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

1. A mat for sanitizing soles of shoes comprising:
a. a top surface;
b. a plurality of grooves along the top surface;
c. a UV light source within each groove, wherein the UV light source is activated upon application of pressure thereon, where each groove is beveled to receive the UV light source;
d. a first side;
e. a second side, where the first side and second side are beveled; and
f. a bottom surface.

2. (canceled)

3. (canceled)

4. A method for sanitizing soles of shoes comprising the steps of:
a. creating beveled groove openings in a mat;
b. inserting a UV light source in each groove;
c. stepping onto the mat with a target pair of shoes of a user;
d. activating a portion of the UV light sources while on the mat, where the portion of the UV light sources are activated by the soles of the target pair of shoes, wherein said user stands on the mat for a predetermined time period.
